(19) United States
(12) Patent Application Publication
(10) Pub. No.: US 2026/0147868 A1
(43) Pub. Date: May 28, 2026

(54) SECURE IDENTITY UTILIZATION PLATFORM

(71) Applicant: Kenneth Bagan, Highland Park, IL (US)

(72) Inventor: Kenneth Bagan, Highland Park, IL (US)

(21) Appl. No.: 18/959,415

(22) Filed: Nov. 25, 2024

(51) Int. Cl. G06F 21/44; G06F 1/16; G06F 21/32; G16H 40/67; H04W 4/029

(52) U.S. Cl. CPC G06F 21/44; G06F 1/163; G06F 21/32; G16H 40/67; H04W 4/029

(57) ABSTRACT

A process for securely enhancing user health via a wearable mobile electronic device is provided. The process entails detecting the user at the device, taking a user biometric reading at the device, identifying the user via the biometric reading, gathering user health-related data at the device, evaluating the gathered user health-related data to determine if the gathered user health-related data evidences a condition of concern or an immediate danger to the user, notifying the user and transmitting a notification to a health care provider if a condition of concern is detected and transmitting a first responder notification if the condition poses an immediate danger to the user, determining at the device whether the user health-related data is to be transmitted to another site and determining at the device whether the user health-related data requires a user display action, and if so executing such an action, and otherwise entering a standby mode.

### TECHNICAL FIELD

The present disclosure is related generally to electronic devices for health and security enhancement and, more particularly, to systems and methods for securely enhancing user health and security via wearable mobile electronic devices.

### BACKGROUND

In a great many scenarios, it is important to individuals as well as to entities and institutions, that individuals can easily and accurately identify themselves. This is true for purposes of access to funds, facilities, events, equipment and so on, as well as for purposes of health tracking, medical treatment and so on.

Although passwords and photo IDs have been used to attempt to meet these needs, neither of these mechanisms is both highly convenient for users and substantially fool-proof. Indeed, over 68 million activity trackers and fitness watches are sold annually without ID security, and most people cannot remember passwords or fail to keep them secure.

Before proceeding to the remainder of this disclosure, it should be appreciated that the disclosure may address some or all of the shortcomings listed or implicit in this Background section. However, any such benefit is not a limitation on the scope of the disclosed principles, or of the attached claims, except to the extent expressly noted in the claims.

Additionally, the discussions of technology in this Background section is reflective of the inventors' own observations, considerations, actions and/or thoughts, and are in no way intended to be, to accurately catalog, or to comprehensively summarize any prior art reference or practice. As such, the inventors expressly disclaim this section as admitted or assumed prior art. Moreover, the identification or implication herein of one or more desirable courses of action reflects the inventors' own observations and ideas, and should not be assumed to indicate an art-recognized desirability.

### BRIEF DESCRIPTION OF THE SEVERAL VIEWS OF THE DRAWINGS

While the appended claims set forth the features of the present techniques with particularity, these techniques, together with their objects and advantages, may be best understood from the following detailed description taken in conjunction with the accompanying drawings of which:

FIG. 1 is a simplified illustration of a device for use within an embodiment of the invention;

FIG. 2 is a schematic diagram of a device usable within embodiments of the invention;

FIG. 3 is a schematic diagram of the network operating environment of the device within an embodiment of the invention;

FIG. 4A is a flow chart showing a process for securely enhancing user health via a wearable mobile electronic device according to an embodiment of the disclosed principles;

FIG. 4B is a continuation of the flow chart of FIG. 4A according to an embodiment of the disclosed principles; and

FIG. 5 is a flow chart showing a process of access, authorization, and/or security tracking and usage via a wearable mobile electronic device according to an embodiment of the disclosed principles.

### SUMMARY

In an embodiment, a process a wearable sensing and communication device is provided having a device module, an attachment portion affixed to the device module, the attachment portion being configured to attach the attachment portion and device module to a user, a bio sensor associated with the device module and the attachment portion, configured to collect biometric data from the user and identify the user based on the collected biometric data.

The wearable sensing and communication device attachment portion may include a clasp that is disengageable only via the use of two hands, thus more permanently securing the wearable device in place on an at-risk user. In a further aspect, the at-risk user may be a child or an at-risk adult such as an elderly adult.

In a further embodiment, the device module is configured to locate the identified user, e.g., via a positioning portion having a GPS module. The wearable sensing and communication device may also include an alarm capability to convey an alarm to the user, for example.

In a further embodiment, the wearable sensing and communication device includes activity sensors and acts as an activity tracker for documenting user movement. In this regard the device module may be registered with a health provider to track user adherence to an exercise regimen, or to a contest provider to facilitate user participation in a health-related contest. In the latter context, the identity capability of the device module is used to prevent fraud in the health-related contest. In an embodiment, the health-related contest is an employer wellness program and in another embodiment, the health-related contest is an insurance-sponsored wellness program. With respect to the exercise regimen, this may be, for example, a cardiac rehab regimen or a university health regimen.

In an embodiment, the user is a hospital patient and the wearable sensing and communication device is affixed to the user upon admission to the hospital.

In a further embodiment, the wearable sensing and communication device further includes a security module configured to transmit a coded user identity to a lock adapted to unlock upon receipt from the device module of a certain preapproved user identity. Thus, the device may, for example, allow authorized entry to a vehicle and/or activate the ignition of a vehicle.

In a further embodiment, the security module is configured to transmit a coded user identity to a point of sale unit to allow or disallow a purchase based on user ID. Similarly, the security module may transmit a coded user identity to a security checkpoint or ticketing checkpoint to allow or disallow passage of the user based on user ID.

### DETAILED DESCRIPTION

Before presenting a detailed discussion of embodiments of the disclosed principles, an overview of certain embodiments is given to aid the reader in understanding the later discussion. Although many companies have attempted to provide users with useful and helpful health data in a convenient manner, such attempts have not been truly successful due to various shortcomings. For example, wider connectivity and data sharing can inure to the user's benefit via data pooling and the resultant inferences, e.g., via AI. However, users who fear misuse of their personal health data may be reluctant to allow their data to be shared, or may be reluctant to even gather such data in the first place. Moreover, controls put in place to ensure data privacy, e.g., passwords, assumptions regarding the user of a device, and other controls are easy to either forget or circumvent.

In embodiments of the disclosed principles, a mobile communications device such as a phone, tablet, watch or band is used to gather user biometric data for identification purposes and for health purposes. In further embodiments, other devices are used to collect user-identifying information. For example, a user's skin pattern or vein pattern may be used for identification purposes, while a blood pressure reading may be taken by the same device. Data gathered in this manner is more helpful to the user because it can be placed in the context of the same user's prior readings. For example a given blood pressure reading may be normal in one user's case and alarming in another user's case, depending upon each user's prior readings.

Scenarios in which convenient automatic user identification may be used abound, but may include, for example, driver ID (e.g., tracking car in an accident, identifying user preexisting conditions), airport area access, linking user to prior purchase, entrance to sporting events, cultural event and festivals, retail purchasing, age verification, vaccination verification, auto check-in for lab, procedure or surgery, inpatient verification for medication or procedure, home security, schools security, preventing welfare fraud, entry to secure facilities, entry to resident facility, test taking ID, link to living will, tracking/managing foreign workers, tracking and managing persons on bail, tracking patient presence at doctor's office or telemedicine meeting, remote illness check and verification, health program reward confirmation, medical data access, individual and team standings on devices, baseline readings, medical device authorization and readings (e.g., blood sugar, radiation, dialysis), managing drug interactions, and so on.

As can be seen, the personal ID system described herein can be beneficially applied both in a health context and a security/control context. That is, many uses provide a route to improved user health, while other uses allow economic or security benefits via verification of user identify and status.

The mechanism used to identify the user may vary depending upon the type of base device being used. For example, bathmats and scales may be equipped with biometric ID capability via footprint ID or skin print ID. In an embodiment, the biometric ID may be gathered via the user's DNA in cases where such DNA is available in ordinary usage. Other devices such as hairbrushes and toothbrushes may additionally or alternatively be used to gather health data and biometric ID data. Fingerprints, retinal patterns, vein patterns and unique heart wave forms also provide means of biometric ID.

With respect to a mat, standing scale or sit-down device, the device may also glean body fat information, e.g., via conductance testing and the like. These tests may be linked to a wearable or other device on the user's person to allow biometric ID in the manner discussed above in connection with such measurements. The sit down device may also allow oxygenation testing via an included oximeter. This is an especially useful parameter to track for individuals with chronic compromised lung function.

To allow a universal weigh-in for weight loss programs such as WEIGHT WATCHERS and others, an initial check-in may be made with a doctor or other health care practitioner, and linked to the user's ID, such that future biometrically-ID'd check-ins will be reliable and secure.

A wearable as described herein also allows secure and verified activity tracking. In particular, since the wearable can ID the user via one or more of the biometric ID methods disclosed herein, the subsequent or contemporaneous activity can be securely associated with the user. In this way, a program sponsor may confidently credit each user with the activity shown on their respective wearable without fear of cheating.

If blood pressure is a health measure of interest in a health care or competitive scenario, cuffless blood pressure measurements may be made to increase user convenience and compliance, while also reducing equipment costs and maintenance. In addition, the absence of a cuff allows such measurements to be taken continuously or frequently, building a better data base for the user or their health care practitioner. Cuffless measurements may include any of blood pressure, pulse, body fat and blood oxygen.

In conjunction with cuffless blood pressure measurements or otherwise, a patch may be used for one or both of health parameter measurement and biometric ID-related measurements. For example, a patch may be used to detect a condition or state, dispense a pharmaceutical substance, and/or take a measurement, such as vein print or fingerprint, usable to biometrically identify the user.

In a further embodiment, the Positive ID device may be a smartwatch having a health parameter measurement and biometric ID capabilities built-in. For example, a smartwatch may be used to take an ECG, which can indicate whether the user's heart is beating normally or abnormally. For example, such a device can detect indications of atrial fibrillation, irregular heartbeat and other improper rhythms that can precede more serious events such as clots, strokes, or heart attack. Other detectable conditions include such diseases as Parkinson's disease and Malaria.

Other measurements may be inferential rather than direct. For example, a wearable or smartphone may be equipped with inertial sensors that determine an aspect of user movement such as gait, stability, and even coordination or symmetry of movement. For example, a user may be prompted to repeat a symmetrical movement while the wearable or smartphone detects any asymmetry of movement, indicative of a stroke. Wearables and smartphones can also be used to measure blood flow, e.g., via photometric analysis, and may measure other health parameters via direct optical observation. The former may be used to determine general circulatory health or readiness for surgery, while the later can be used to detect or investigate ocular abnormalities. Indeed, direct optical observation may even be used to measure the color of bodily fluids, such as urine, to detect indicators of a disease or condition such as cancer.

In a further embodiment, external sensors, connectable to the smartphone or wearable may be used to detect specific diseases such as HIV. Moreover, a microphone built into the wearable or smartphone may be used to analyze user sounds for diagnostic purposes. For example, the sound of a user's cough can be used to diagnose respiratory disease.

In addition to taking data and measurements, the described system may also provide information in various forms. In particular, not only does the system provide health data when appropriate, but the system may also provide enhanced sense experiences for entertainment or advertising. Thus, for example, the device may provide visual 3D events and may even provide 4D experiences by providing other sensory data such as scents and motions. In an embodiment, a smell chip or other scent sensor is included in or attached to the device.

With this overview in mind, and turning now to a more detailed discussion in conjunction with the attached figures, the techniques of the present disclosure are illustrated as being implemented in or via a suitable device environment. The following device description is based on embodiments and examples within which or via which the disclosed principles may be implemented, and should not be taken as limiting the claims with regard to alternative embodiments that are not explicitly described herein.

As discussed above, embodiments of the invention pertain to biometric measurement devices coupled to a wide area network such as the Internet. The link to the network may be implemented by way of wired or wireless connections or a combination thereof, and while high speed connections such as DSL are preferred, slower connections may instead be used.

The various innovations described herein may be implemented in a stationary machine for some uses, in a mobile device for other uses, and via a combination of a stationary unit and a mobile unit for yet other uses. As such, the figures with show both types of device, and certain combination thereof.

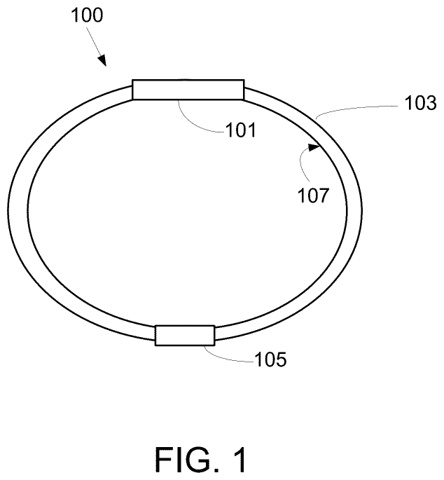

FIG. 1 is a simplified illustration of a device for use within an embodiment of the invention. The illustrated device 100 is a wearable band device including an electronic device 101, band 103 and clasp 105. The clasp 105 may be configured to require two hands to unclasp as will be further discussed later herein. The device 100 includes a biometric sensor to identify the user, such as any of the BioID mechanisms mentioned elsewhere herein., In an embodiment, the inner surface 107 of the band 105 comprises a vein reader to identify the user and may also comprise one or more health sensors such as blood pressure sensors, glucose sensors and so on.

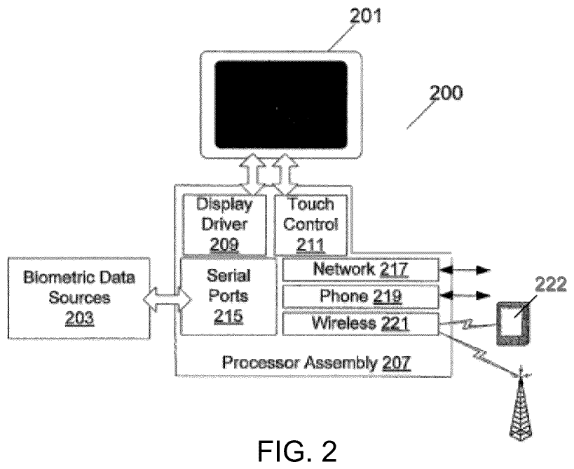

As discussed above, the device 100 also comprises a link to a network. This and other detailed aspects of the device are illustrated schematically in FIG. 2. In particular, FIG. 2 is a schematic diagram of a device usable within embodiments of the invention. The device 200 comprises biometric data sources 203 and a display 201.

These elements 201, 203 are linked, typically by wired connections, to a processor assembly 207. The display 201 interfaces with the processor assembly 207 via a display driver 209 and a touch control module 211. The touch control module 211 receives and processes touch screen inputs from the display 201. The biometric data sources 203 interfaces with the processor assembly 207 via ports 215.

The processor assembly 207 also comprises data links to external data sinks/sources. For example, in the illustrated embodiment of the invention, the processor assembly 207 comprises a network communication module 217, a phone communication module 219, and a wireless communication module 221. As will be discussed in greater detail below, the wireless communication module 221 allows connectivity to a cellular network and/or to local wireless devices via a short range protocol such as Bluetooth. The network communication module 217 provides connectivity (wired or wireless) to one or more networks such as a local area network (LAN) and the Internet.

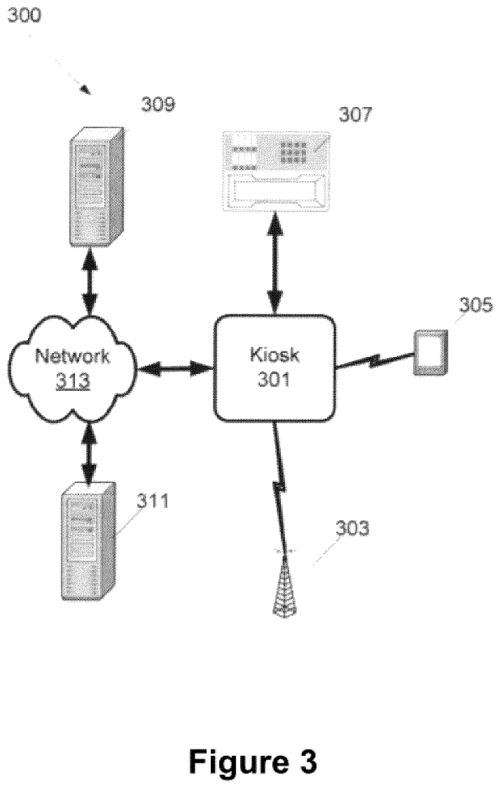

FIG. 3 is a schematic diagram of the network operating environment 300 of the device within an embodiment of the invention. As illustrated, the device 301 is communicably linked to a phone system 307, such as via ordinary phone lines. The device 301 is also linked wirelessly to a cellular network 303 and a local wireless device 305, e.g., a Bluetooth equipped device. The local wireless link allows the user to use their own phone to communicate to or from the device as well as to place a call through the device to a customer service representative or advisor.

Finally, the device 301 is linked via a network 313 to a number of networked data sources/sinks, e.g., servers 309, 311. In an embodiment of the invention, server 309 is an operator server of the company or other entity responsible for the device. For example, LifeClinic® is the operator of devices that are placed in thousands of locations across the country. In this embodiment of the invention, the other server 311 is associated with a sponsor or other entity interested in receiving data from the devices as will be discussed in greater detail below.

The data transferred over the network links to/from one or both of servers 309, 311 to/from the device 100 includes in an embodiment of the invention any or all of the communication indicated herein, including but not requiring: waiver, identity, readings, demographics and other data from the device 100; instructions, video, communications, prior readings, and advertisements from the server(s) 309, 311.

The way in which the device of FIGS. 1-3 is used depends to some extent on the environment in which it is used. One exemplary environment is a mall network, i.e., a number of devices in one or more malls linked via the Internet or other network to operator and/or sponsor servers. Another exemplary environment is a university network, i.e., a number of devices in one or more universities linked via the Internet or other network to operator and/or sponsor servers. One or more universities may additionally be divided into subnets such as a student union network, a recreation center network and a student health center network. Another exemplary environment is a hospital network, i.e., a number of devices in one or more hospitals linked via the Internet or other network to operator and/or sponsor servers. Hospital devices may be provided in many areas, such as emergency rooms, gift shops, and rehabilitation departments. Another exemplary environment is a doctor's office network, i.e., a device in one or more doctor's offices linked via the Internet or other network to operator and/or sponsor servers. Yet another exemplary environment is an airline club network, i.e., a device in one or more Airline Clubs linked via the Internet or other network to operator and/or sponsor servers. A broader example of the foregoing network is a traveler's network in which devices as described above may be placed in locations frequented by travelers, e.g., train stations, airports, bus stations, etc. Yet another exemplary environment is a work site network, i.e., a device in one or more work sites linked via the Internet or other network to operator and/or sponsor servers. This network would allow employees to access their readings at home and to print a history of readings. Employee readings could also be emailed with regard to health results to a user's health care provider. Yet another exemplary environment is a health club network, i.e., a device in one or more health clubs linked via the Internet or other network to operator and/or sponsor servers. Yet another exemplary environment is a not-for-profit environment. Examples would include devices in religious institutions such as churches, mosques, and temples to promote religious or charitable causes, products, or activities. Other exemplary environments include convention centers (e.g., to provide health information), municipal buildings (City Halls, Fire Departments, etc., e.g., so customers can register for the local zoo membership, apply for car registration, etc.), government groups or centers, museums (e.g., to sell museum memberships and gift shop items), etc. Another environment in which a device system according to the invention can be beneficially employed is within a hotel or hotel chain.

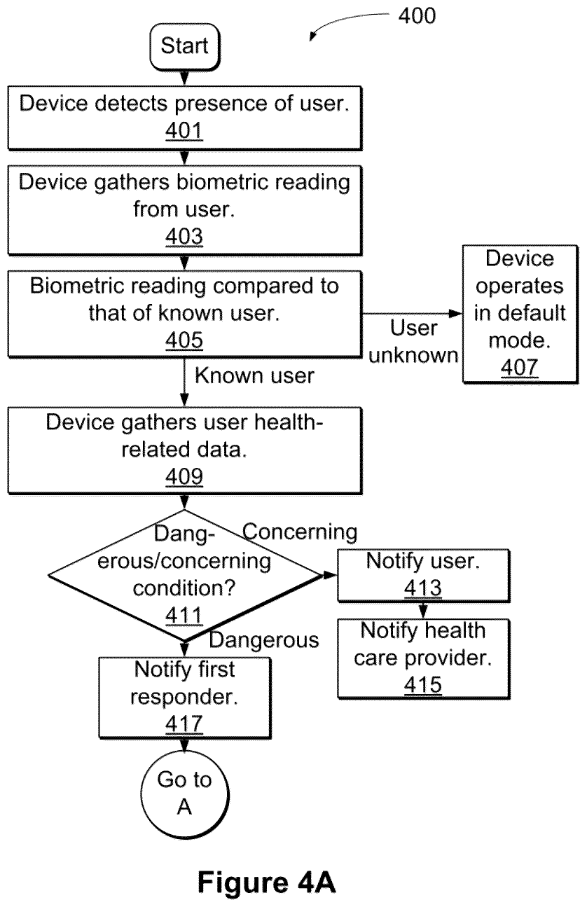

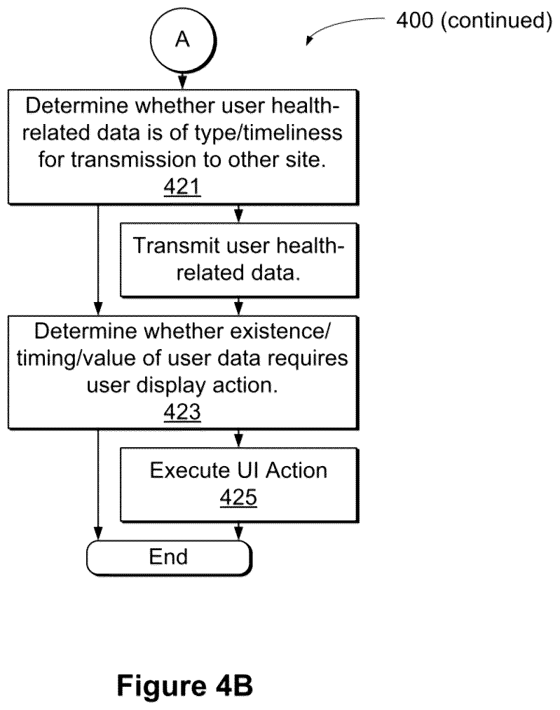

Having discussed various types of health device networks and environments above, the details of certain types of exchanges facilitated by certain embodiments of the invention will now be discussed. FIGS. 4A-4B show a flow chart of a process 400 for securely enhancing user health via a wearable mobile electronic device.

At stage 401 of the process 400, the device detects the presence of the user. This may be accomplished via capacitive or touch detection, or via alternative means such as by detecting the presence of a device such associated with the user, such as a Bluetooth head set.

Having detected the user, the device then gathers a biometric reading relative to the user at stage 403. As noted above, this biometric reading may include a fingerprint, palm print, footprint, toe print, vein print, voice print, retinal print, heart rhythm, gait, DNA and so on. At stage 405, the gathered biometric reading is compared to a stored biometric reading of a known authorized user. If the collected biometric reading matches the stored biometric reading to an acceptable degree (e.g., a 95% match between fingerprints), the device identifies the user in accordance with the stored biometric reading.

Otherwise, the process 400 branches to stage 407, wherein it operates in accordance with a default mode, which may still encompass non-verified health related activities. It should be noted that the identification of the user herein is not necessarily an authorization of the user to operate the device as to any aspect other than those relevant to the process 400. That is, for example, being identified for health purposes herein does not imply authorization for other purposes such as checking messages, executing financial transactions, and so on. Unrelated functions such as communications and financial applications will continue to operate according to their default requirements.

At stage 409, the device gathers user health-related data such as blood-pressure, user weight, pulse rate, body fat and blood oxygen, body temperature, user activity or other parameters as will be appreciated by those of skill in the art. The gathered user health-related data is first evaluated at stage 411 to determine if the data evidences a condition of concern or an immediate danger to the user. For example, the gathered user health-related data could potentially reveal a current or impending health condition such as a stroke, heart attack, illness, or other condition of concern.

If a condition of concern is detected at stage 411, the process 400 notifies the user of the device at stage 413 and transmits a notification to a health care provider associated with the user at stage 415, given the user ID and stored user data on the device. If the condition is not just of concern but further poses an immediate danger, the device transmits a first responder notification at stage 417. This may be, for example, an alert and request for service sent to local emergency medical services or other first responder.

At stage 419, the device stores the user health-related data with other user health-related data on the device or at a remote site associated with the device. The device also determines at stage 421 (FIG. 4B) whether the user health-related data is of the type or timeliness to be transmitted to another site, such as a hospital remote monitoring site, a health contest management site, an insurer reward site and so on. Such data may be, for example, data showing completion of an assigned exercise task such as a walk or workout, data showing a periodically logged measurement such as user weight or blood pressure, data showing attendance at a required event, such as a cardiac rehabilitation session, data showing the presence or absence of a disease or condition, such as influenza, and so on.

The device determines at stage 423 whether the existence, timing or value of the user health-related data requires a user display action such as an acknowledgement, a warning, a listing of user progress, a user contest standing or other UI action. If such an action is required, it is executed at stage 425. In an embodiment, the health contest is a cycling contest including stationary cycling, such as on a stationary bike or nonstationary cycling.

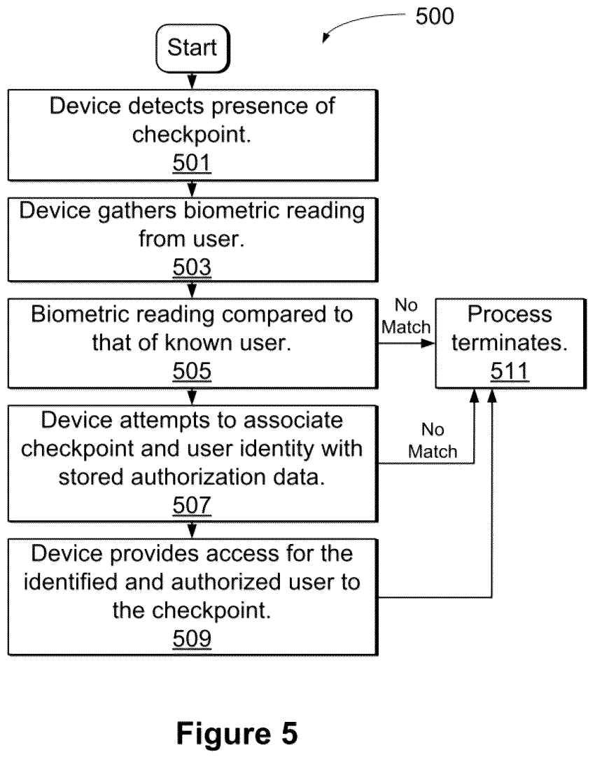

Turning to FIG. 5, this figure illustrates a process of access, authorization, and/or security tracking and usage. The process 500 starts at stage 501, wherein the wearable user device detects a checkpoint, such as a gate, portal, entry door, barrier etc. Detection of the checkpoint may be made automatically via detection of an emitted RF signal, an audio signal, the presence of auto-recognized signage such as QR codes and so on.

Upon detection of the checkpoint, the device checks the user identity by gathering a biometric reading relative to the user at stage 503 and identifies the user at stage 505 by comparing the biometric reading to analogous stored biometric readings of one or more known users. As noted above, the biometric readings may include a fingerprint, palm print, footprint, toe print, vein print, voice print, retinal print, heart rhythm, gait, DNA and so on. If the collected biometric reading matches a stored biometric reading to a predetermined extent, the device identifies the user in accordance with the stored biometric reading. If the user cannot be identified, the process 500 diverts to stage 511 and terminates.

Otherwise, the device attempts to associate the identity of the checkpoint and the identity of the user with stored authorization data at stage 507. If no authorization data is found for the identified user and the relevant checkpoint, the process 500 diverts to stage 511 and terminates. The checkpoint authorization may originate from a prior payment, such as for a concert, game, museum, etc., or may be tied to a security clearance, e.g., for an airport, restricted area, etc., or may be linked to a required condition, e.g., vaccination, lack of illness, outstanding warrants, owner of the checkpoint (in the case of a vehicle), and so on.

If authorization data is found for the identified user and the relevant checkpoint, the device provides access to the user at stage 509 by displaying a bar code, QR code, pass number, etc. used to gain entry, or emitting an access message via NFC or Bluetooth allowing access or entry.

The mobile device comprises at its core a processor linked to various other system components. The processor may be of any suitable type including one or more microprocessors, programmable logic controllers, and so, and may be implemented within a personal computer, workstation, or other computing device, or may be integrated into the device in a customized form. The processor is linked to a one or more verification input devices, e.g., a fingerprint scanner, a camera, or a retinal scanner 605. It will be appreciated that other types of verification devices may be used instead or in addition to those listed.

The processor is also linked to one or more internal or external, local or remote databases, e.g., a fingerprint database, a facial feature database, a retinal feature database, and an image database. The processor is also linked to other components of the device including a user interface, such as a screen, speaker, keyboard, buttons, etc. Additional components include biometric data sources for devices that measure such information. The biometric data gathered can include parameters such as user weight, blood pressure, pulse rate, body fat and blood oxygen, or other parameters as will be appreciated by those of skill in the art.

The processor is further linked to a wireless communication interface which provides a mechanism for wirelessly communicating with nearby electronic devices. For example, the wireless communication interface is adapted in an embodiment to wirelessly call a user's cell phone and connect the user via wide area communications facilities 625 to a call center or emergency personnel.

The fingerprint scanner operates in a manner familiar to those of skill in the art, either alone or in conjunction with processes executed on processor or other component. The fingerprint scanner obtains an image of a user's finger compares this image to pre-scanned images, e.g., in database, to determine the identity of the user by determining whether the patterns of ridges and valleys in the images match. Although the fingerprint scanner may be of any suitable construction, it will be appreciated that fingerprint scanners generally utilize either optical or capacitive technologies.

Optical scanners generally employ a charge coupled device (CCD) to capture the print image. Typically, an analog-to-digital converter in the scanner system processes the analog electrical output of the CCD to generate a digital image. The fingerprint scanner may comprise a light source such as an LED array to facilitate image acquisition. Much like optical scanners, capacitive fingerprint scanners also generate an image of a fingerprint. However, rather than using photons, they employ changes in capacitance across the print. One advantage of capacitance type sensors is that they cannot be manipulated via an image of a fingerprint. Additionally, capacitive scanners can be made more compact than optical devices.

For purposes of facial recognition, the camera captures an image of the user's face and uses this image to verify the user's identity. Human faces have certain distinguishable features or landmarks. For example, there are distinct dips and rises that make up different facial features. Depending upon the algorithm used, as many as 80 landmarks may be identified and used for recognition.

Examples of landmarks include the distance between a user's eyes, the width of a user's nose, depth of a user's eye sockets, shape or location of a user's cheekbones, shape or location of a user's jaw line, and the shape or location of a user's chin. Features of the landmark points are measured and translated to a unique numerical code or “faceprint” that represents the particular face. Pre-scanned faces, or related representations such as face prints, are stored to facilitate later comparison for validation. Registration of a user's faceprint to be used for later verification may occur upon the user's first use of a device system as described herein, or may be obtained elsewhere, e.g., at a registration desk.

The retinal scanner obtains an image of the user's retina and compares this to prior stored scans to verify a user's identity. Retinal scanners are commercially available, and operate in a manner similar to that described above, i.e. by matching a retina of interest, or characteristics thereof, to a validation image of a retina or to characteristics of such an image.

It will be appreciated that other identity verification techniques may be used additionally or alternatively to verify a user's identity. Other techniques include, but are not limited to, voiceprint recognition, vein pattern recognition, iris recognition, etc.

One area where significant incentives may be provided and hence where enhanced identity resolution is important is the area of insurance incentives. In particular, insurers may enroll with a device system host to receive user medical information from such devices and to provide targeted advertising and/or incentives through the devices. In particular, a user may use any one of the global network of devices to take a measurement of interest, e.g., weight, body fat percentage, or blood pressure, and this information will be transmitted to the enrolled insurer. In an embodiment of the invention, the device displays a list of enrolled insurers to the user, and the user selects the appropriate company name.

If the user's current and prior measurements show a positive trend (e.g., losing body fat) at an acceptable rate (e.g., 2 pounds per week) or in an acceptable amount (e.g., 10 pounds), then the insurer will provide a reward to the user. Rewards can consist of rebates, gift cards, reduced insurance rates, and so on. Thus, users may be tempted to cheat to obtain the incentives, i.e., by having a friend in better health sit in as the user. To avoid fraud and cheating, it is important that the insurer be able to verify that the subscriber being rewarded has indeed made the appropriate improvements.

With respect to contests, the user may be enrolled in a health contest via the device or devices with another person or persons, e.g., work site personnel, school personnel or students, gym members, and so on. In this case, the user's positively identified results can be logged with the readings of others users to determine a contest winner at the conclusion of the contest.

In a further embodiment, a device, device system, or device network as described above, is used to facilitate a Cardiac Rehabilitation Program (CRP). It will be appreciated that after a heart attack or other heart health event, the acute phase of treatment ends very early and gives way to a longer but still important rehabilitation process. In this CRP, the user is instructed to undertake certain activities while also being monitored for their heart response and conditioning. Often, this process takes place on a session basis, with the user coming into a central location multiple times per week to perform activities while being monitored.

However, traditional CRPs operating as discussed above have a number drawbacks built into their structure. For example, many patients do not comply with the rehab requirements, primarily by missing one or more sessions. Others may drop out of rehab entirely. It has been shown that patients who fail to comply with rehab instructions are more likely to be readmitted, resulting in greater costs to the system as well as negatively impacting hospitals'performance statistics.

In this embodiment of the described techniques, Bio-ID modified devices are provided, e.g., at no cost (by Medicare, Insurance Company, etc.) to CRP-eligible patients. The provider may require the patient to follow program requirements as a condition of providing use of the Bio-ID modified device. Preferably, cardiac rehabilitation personnel instruct each patient regarding the mechanics (connecting the devices, charging them and so on) and the program goals and requirements.

The Bio-ID modified device may be or have a similar form factor to a smartwatch or other wearable device. The modifications allow to the device to operate as discussed above, e.g., ensuring patient ID via Biometric Positive ID (skin ID for example), monitoring user activities and/or health parameters (via device sensors and patient parameter sensors), and transmitting relevant health data securely to the cardiac rehabilitation center for monitoring purposes. In this way, the user is monitored and may be compliant with—and involved in—the CRP regardless of whether they are physically at the cardiac rehabilitation center at any given time. In a further embodiment, the modified device also monitors compliance with other CRP activities, such as the watching of educational videos and so on.

In a further embodiment, the patient may still be required to periodically check-in at the cardiac rehabilitation center. The device may also allow contact from the cardiac rehabilitation center to the patient in the event that the patient is to be notified of program changes or compliance issues.

Depending on program preference, the device may be returned to the sponsor or cardiac rehabilitation center at the end of the CRP. Alternatively, the device may be retained to act as a safety and health device as discussed elsewhere herein, e.g., to alert upon sensing dangerous conditions, to engage in contests, to allow workplace safety functions and so on. The retention of the device by a patient may also be used to provide further health advice, advertising and public service announcements.

To summarize and reiterate, In an embodiment, a process a wearable sensing and communication device is provided having a device module, an attachment portion affixed to the device module, the attachment portion being configured to attach the attachment portion and device module to a user, a bio sensor associated with the device module and the attachment portion, configured to collect biometric data from the user and identify the user based on the collected biometric data.

The wearable sensing and communication device attachment portion may include a clasp that is disengageable only via the use of two hands, thus more permanently securing the wearable device in place on an at-risk user. In a further aspect, the at-risk user may be a child or an at-risk adult such as an elderly adult.

In a further embodiment, the device module is configured to locate the identified user, e.g., via a positioning portion having a GPS module. The wearable sensing and communication device may also include an alarm capability to convey an alarm to the user, for example.

In a further embodiment, the wearable sensing and communication device includes activity sensors and acts as an activity tracker for documenting user movement. In this regard the device module may be registered with a health provider to track user adherence to an exercise regimen, or to a contest provider to facilitate user participation in a health-related contest. In the latter context, the identity capability of the device module is used to prevent fraud in the health-related contest. In an embodiment, the health-related contest is an employer wellness program and in another embodiment, the health-related contest is an insurance-sponsored wellness program. With respect to the exercise regimen, this may be, for example, a cardiac rehab regimen or a university health regimen.

In an embodiment, the user is a hospital patient and the wearable sensing and communication device is affixed to the user upon admission to the hospital.

In a further embodiment, the wearable sensing and communication device further includes a security module configured to transmit a coded user identity to a lock adapted to unlock upon receipt from the device module of a certain preapproved user identity. Thus, the device may, for example, allow authorized entry to a vehicle and/or activate the ignition of a vehicle.

In a further embodiment, the security module is configured to transmit a coded user identity to a point of sale unit to allow or disallow a purchase based on user ID. Similarly, the security module may transmit a coded user identity to a security checkpoint or ticketing checkpoint to allow or disallow passage of the user based on user ID.

The described principles enable many innovative health and activity monitoring solutions through different modalities. These include Smartwatch health monitoring with Skin-ID bio-identification, Activity Tracking Devices (straps and bands) with Skin-ID, blood pressure device with facial recognition Bio-ID, pulse rate device with facial recognition, Weight Scale Device with Facial Recognition, Pulse Oximeter Device with Fingerprint, Glucose Testing Device, Temperature Device, EKG Device and vital sign monitoring via TTK (Triple Test Kit), e.g., with facial recognition and/or fingerprint ID on one or more of several cooperating devices (Bio-ID Scale, cuff, pedometer, etc.).

For temperature readings, a Bio-ID thermometer may use saliva/DNA to biometrically identify the user. Similarly, for the glucose meter, the user's blood/DNA may be used to biometrically identify the user. And finally, with respect to EKG, such as via available consumer-grade EKG devices with two finger sensor pads, fingerprint analysis can be used to biometrically identify the user.

With respect to tracking via fitness products, several current fitness bands/watched include oximeter capabilities, which can be augmented via skin/vein biometric identification to biometrically identify the user. In this way, important health and activity data can be uploaded to authorized healthcare entities (with user's permission) for monitoring, alerts and so on. The same information may also be valuable for public health, e.g., via an educational website on Coronavirus, when oximeter readings can be indicative of a disease process such as a coronavirus infection. In an embodiment, a fingertip pulse oximeter with Fingerprint ID is able to both ID the user and check or verify oximeter data.

It will be appreciated that various systems and processes have been disclosed herein. However, in view of the many possible embodiments to which the principles of the present disclosure may be applied, it should be recognized that the embodiments described herein with respect to the drawing figures are meant to be illustrative only and should not be taken as limiting the scope of the claims. Therefore, the techniques as described herein contemplate all such embodiments as may come within the scope of the following claims and equivalents thereof.

1. A wearable sensing and communication device comprising:
a device module;
an attachment portion affixed to the device module, the attachment portion being configured to attach the attachment portion and device module to a user;
a bio sensor associated with at least one of the device module and the attachment portion, the bio sensor being configured to collect biometric data from the user and being linked to the device module; and
the device module being further configured to identify the user based on the collected biometric data.

2. The wearable sensing and communication device in accordance with claim 1, wherein the attachment portion includes a clasp and wherein the clasp is disengageable only via the use of two hands, thus more permanently securing the wearable device in place on an at-risk user.

3. The wearable sensing and communication device in accordance with claim 2, wherein the at-risk user is a child.

4. The wearable sensing and communication device in accordance with claim 2, wherein the at-risk user is am at-risk adult.

5. The wearable sensing and communication device in accordance with claim 1, wherein device module is configured to locate the identified user.

6. The wearable sensing and communication device in accordance with claim 5, wherein device module includes a positioning portion configured to locate the device.

7. The wearable sensing and communication device in accordance with claim 6, wherein positioning portion comprises a GPS module.

8. The wearable sensing and communication device in accordance with claim 1, further comprising an alarm capability.

9. The wearable sensing and communication device in accordance with claim 1, wherein with device module includes activity sensors and acts as an activity tracker for documenting user movement.

10. The wearable sensing and communication device in accordance with claim 9, wherein with device module is registered with a health provider to track user adherence to an exercise regimen.

11. The wearable sensing and communication device in accordance with claim 9, wherein with device module is registered with a contest provider to facilitate user participation in a health-related contest.

12. The wearable sensing and communication device in accordance with claim 11, wherein the identity of the user by the device module is used to prevent fraud in the health-related contest.

13. The wearable sensing and communication device in accordance with claim 11, wherein the health-related contest is an employer wellness program.

14. The wearable sensing and communication device in accordance with claim 11, wherein the health-related contest is an insurance-sponsored wellness program.

15. The wearable sensing and communication device in accordance with claim 10, wherein the exercise regimen is a cardiac rehab regimen.

16. The wearable sensing and communication device in accordance with claim 10, wherein the exercise regimen is a university health regimen.

17. The wearable sensing and communication device in accordance with claim 1, wherein the user is a hospital patient and the wearable sensing and communication device is affixed to the user upon admission to the hospital.

18. The wearable sensing and communication device in accordance with claim 1, wherein the device module further comprises a security module configured to transmit a coded user identity to a lock adapted to unlock upon receipt from the device module of a certain preapproved user identity.

19. The wearable sensing and communication device in accordance with claim 18, wherein the lock allows entry to a vehicle.

20. The wearable sensing and communication device in accordance with claim 18, wherein the lock activates the ignition of a vehicle.

21. The wearable sensing and communication device in accordance with claim 18, wherein the security module is further configured to transmit a coded user identity to a point of sale unit to allow or disallow a purchase based on user ID.

22. The wearable sensing and communication device in accordance with claim 18, wherein the security module is further configured to transmit a coded user identity to a security checkpoint to allow or disallow passage of the user based on user ID.

23. The wearable sensing and communication device in accordance with claim 18, wherein the security module is further configured to transmit a coded user identity to a ticketing checkpoint to allow or disallow passage of the user into an event based on user ID.
